(19) United States
(12) Patent Application Publication
(10) Pub. No.: US 2024/0336899 A1
(43) Pub. Date: Oct. 10, 2024

(54) CARBONYL REDUCTASE MUTANT AND APPLICATION THEREOF

(71) Applicants: SHANGHAI INSTITUTE OF PHARMACEUTICAL INDUSTRY CO,. LTD., Shanghai (CN); CHINA STATE INSTITUTE OF PHARMACEUTICAL INDUSTRY CO,. LTD., Shanghai (CN)

(72) Inventors: Shaoxin CHEN, Shanghai (CN); Fuli ZHANG, Shanghai (CN); Jiawei TANG, Shanghai (CN); Guowei NI, Shanghai (CN); Luwen ZHANG, Shanghai (CN); Xiao LIU, Shanghai (CN); Jun YU, Shanghai (CN)

(21) Appl. No.: 18/681,359

(22) PCT Filed: Aug. 5, 2022
(86) PCT No.: PCT/CN2022/110530

§ 371 Date: Feb. 5, 2024

(30) Foreign Application Priority Data
Aug. 5, 2021 (CN) 202110896829.4

(51) Int. Cl. C12N 9/04; C12P 13/00; C12P 13/02

(52) U.S. Cl. CPC C12N 9/0006; C12P 13/001; C12P 13/02; C12Y 101/01184

(57) ABSTRACT

A carbonyl reductase mutant and application thereof are provided. Mutation sites of the carbonyl reductase mutant include the 88th, 142nd, 190th, and 193rd positions of the amino acid sequence shown in SEQ ID NO:1. The carbonyl reductase mutant has higher enzymatic activity than wild-type carbonyl reductases. The enzymatic activity of some carbonyl reductase mutants is 50 times that of the wild-type carbonyl reductases. The carbonyl reductase mutant can cause a compound shown in formula I to carry out a reduction reaction shown in the following formula in a liquid reaction system in the presence of coenzymes, can prepare a compound represented by formula II with a conversion rate greater than 99%, a chiral ee value greater than 99%, and a chiral de value greater than 99%.

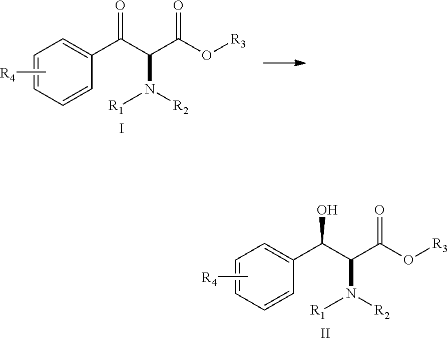

The present application claims the priority for Chinese patent application 2021108968294 with a filing date of Aug. 5, 2021. The present application cites the said Chinese patent application in its entirety.

### TECHNICAL FIELD

The present invention belongs to the field of biocatalytic synthesis and relates to a carbonyl reductase mutant and application thereof.

### BACKGROUND

Compounds containing chiral amino alcohol structures have diverse biological activities and are widely used in the fields of pharmaceutical and chemical, such as Droxidopa for the treatment of Parkinson's disease and hypotension, chloramphenicol with broad-spectrum antimicrobial activity, veterinary drugs Florfenicol and Thiamphenicol, oral drug Elicitorostat for treatment of Gaucher disease, and other clinical candidates with anti-inflammatory, anti-infective, and anti-tumor activities. Among them, chloramphenicol, which was launched in 1949 as a broad-spectrum antibiotic, has a global production mainly concentrated in China, with a production scale of about 3,000 tons. However, with the continuous improvement of environmental protection standards and the increasing cost of treating the three wastes (waste water, waste gas, and waste solid), the existing production process is no longer sufficient to meet the requirements of development. Therefore, there is an urgent need for a more green, economical, and environmentally friendly process route to solve the environmental issues in the existing production process. The biocatalytic method of using carbonyl reductase-mediated dynamic kinetic splitting reduction in the disclosure of Chinese patent application CN111808893A in our laboratory can effectively solve the problems of high pollution, high energy consumption, low efficiency and low quality in the existing production process of chloramphenicol. Therefore, there is an urgent need in the industry for a more efficient and stable carbonyl reductase for the synthesis of chloramphenicol and other chiral amino alcohol drugs.

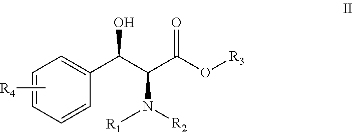

### SUMMARY

The technical problem to be solved by the present invention is to overcome the deficiency of low activity of carbonyl reductase mutants in prior art, and to provide a carbonyl reductase mutant and application thereof. The carbonyl reductase mutant of the present invention has higher enzyme activity compared to the wild-type carbonyl reductase and can be used to catalyze a carbonyl reduction reaction of the compound shown in Formula I

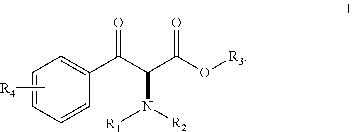

The present invention provides a carbonyl reductase mutant, wherein the mutation site of the carbonyl reductase mutant comprises positions 88, 142, 190, and 193 of the amino acid sequence as shown in SEQ ID NO: 1.

In the present invention, preferably, the mutation site of the carbonyl reductase mutant further comprises one or more positions selected from positions 82, 121, 138, 192, 201, 204, 206, and 207 of the amino acid sequence as shown in SEQ ID NO: 1.

In the present invention, more preferably, the mutation site of the carbonyl reductase mutant further comprises at least 3 positions selected from positions 82, 121, 138, 192, 201, 204, 206, and 207 of the amino acid sequences as shown in SEQ ID NO: 1.

In the present invention, preferably, the mutation site of the carbonyl reductase mutant is selected from any one of the following groups:
(1) the mutation sites of the carbonyl reductase mutant are positions 82, 88, 121, 138, 142, 190 and 193 of the amino acid sequence as shown in SEQ ID NO: 1;
(2) the mutation sites of the carbonyl reductase mutant are positions 82, 88, 121, 138, 142, 190, 193 and 201 of the amino acid sequence as shown in SEQ ID NO: 1;
(3) the mutation sites of the carbonyl reductase mutant are positions 82, 88, 121, 138, 142, 190, 193, 204 and 206 of the amino acid sequence as shown in SEQ ID NO: 1;
(4) the mutation sites of the carbonyl reductase mutant are positions 82, 88, 121, 138, 142, 190, 193, 206 and 207 of the amino acid sequence as shown in SEQ ID NO: 1;
(5) the mutation sites of the carbonyl reductase mutant are positions 82, 88, 121, 138, 142, 190, 193, 201, 206 and 207 of the amino acid sequence as shown in SEQ ID NO: 1;
(6) the mutation sites of the carbonyl reductase mutant are positions 82, 88, 121, 138, 142, 190, 192, 193, 204 and 206 of the amino acid sequence as shown in SEQ ID NO: 1;
(7) the mutation sites of the carbonyl reductase mutant are positions 82, 88, 121, 138, 142, 190, 193, 201 and 204 of the amino acid sequence as shown in SEQ ID NO: 1.

In the present invention, preferably, the amino acid residue at position 82 is mutated from W to L.

In the present invention, preferably, the amino acid residue at position 88 is mutated from F to V, I or S, such as I or V.

In the present invention, preferably, the amino acid residue of position 121 is mutated from V to A.

In the present invention, preferably, the amino acid residue at position 138 is mutated from A to V or L, such as L.

In the present invention, preferably, the amino acid residue at position 142 is mutated from R to M, F, H, or L, such as M.

In the present invention, preferably, the amino acid residue at position 190 is mutated from A to V.

In the present invention, preferably, the amino acid residue at position 192 is mutated from R to M.

In the present invention, preferably, the amino acid residue at position 193 is mutated from S to A.

In the present invention, preferably, the amino acid residue at position 201 is mutated from Y to F.

In the present invention, preferably, the amino acid residue at position 204 is mutated from N to A or G, such as A.

In the present invention, preferably, the amino acid residue at position 206 is mutated from K to H.

In the present invention, preferably, the amino acid residue at position 207 is mutated from K to N.

In the present invention, the mutation sites and types of the carbonyl reductase mutant are shown in Table 1:

TABLE 1

| | Amino acid residue difference from |
|---|---|
| Mutant | SEQ ID NO:1 |
| Mutant 19 | F88V, R142M, A190V, S193A |
| Mutant 21 | F88I, R142L, A190V, S193A |
| Mutant 23 | F88I, R142M, A190V, S193A |
| Mutant 25 | F88I, R142F, A190V, S193A |
| Mutant 27 | F88V, R142L, A190V, S193A |
| Mutant 29 | F88S, R142H, A190V, S193A |
| Mutant 31 | F88V, A138V, R142L, A190V, S193A |
| Mutant 33 | F88I, A138L, R142I, A190V, S193A |
| Mutant 35 | F88I, A138L, R142F, A190V, S193A |
| Mutant 37 | F88I, A138L, R142L, A190V, S193A |
| Mutant 39 | F88V, A138L, R142L, A190V, S193A |
| Mutant 41 | F88I, A138L, R142M, A190V, S193A |
| Mutant 43 | F88V, A138L, R142M, A190V, S193A |
| Mutant 45 | W82L, F88V, A138L, R142M, A190V, S193A |
| Mutant 49 | W82L, F88V, V121A, A138L, R142M, A190V, S193A |
| Mutant 51 | W82L, F88V, V121A, A138L, R142M, A190V, S193A, |
| | Y201F |
| Mutant 53 | W82L, F88V, V121A, A138L, R142M, A190V, S193A, |
| | N204A, K206H |
| Mutant 55 | W82L, F88V, V121A, A138L, R142M, A190V, S193A, |
| | N204G, K206H |
| Mutant 57 | W82L, F88V, V121A, A138L, R142M, A190V, S193A, |
| | K206H, K207N |
| Mutant 59 | W82L, F88I, V121A, A138L, R142M, A190V, S193A, |
| | K206H, K207N |
| Mutant 61 | W82L, F88I, V121A, A138L, R142M, A190V, S193A, |
| | Y201F, K206H, K207N |
| Mutant 63 | W82L, F88V, V121A, A138L, R142M, A190V, R192M, |
| | S193A, N204A, K206H, |
| Mutant 65 | W82L, F88V, V121A, A138L, R142M, A190V, S193A, |
| | Y201F, N204A |

The present invention further provides a preparation method for the compound shown in Formula II, comprising the following steps of: in a liquid reaction system, the compound as shown in Formula I being subjected to the reduction reaction as shown in the following formula in the presence of coenzyme and the aforementioned carbonyl reductase mutant;

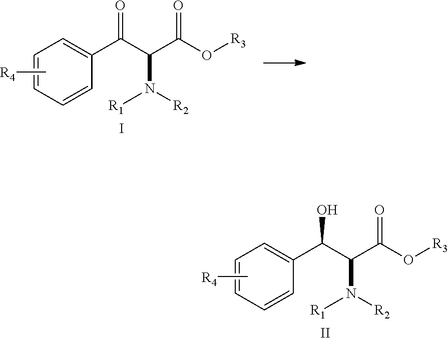

R₁ is H,

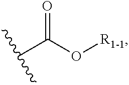

or benzyl;
R₁₋₁ is C₁-C₆ alkyl or benzyl;
R₂ is H,

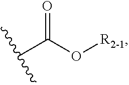

or benzyl;
R₂₋₁ is C₁-C₆ alkyl or benzyl;
R₃ is

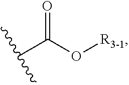

wherein R₃₋₁ is C₁-C₆ alkyl;
R₄ is H, NO₂, halogen, C₁-C₆ alkyl, C₁-C₆ alkoxy, or C₁-C₆ alkyl substituted sulfonyl.

In the present invention, preferably, the halogen is F, Cl, Br or I.

In the present invention, preferably, the C₁-C₆ alkyl is a C₁-C₄ alkyl, such as methyl, ethyl, n-propyl, isopropyl, n-butyl, sec-butyl, isobutyl or tert-butyl.

In the present invention, preferably, R₁ is H,

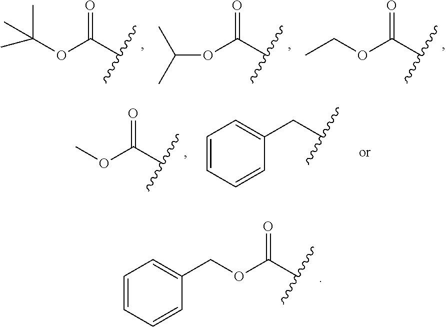

In the present invention, preferably, R₂ is H,

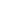

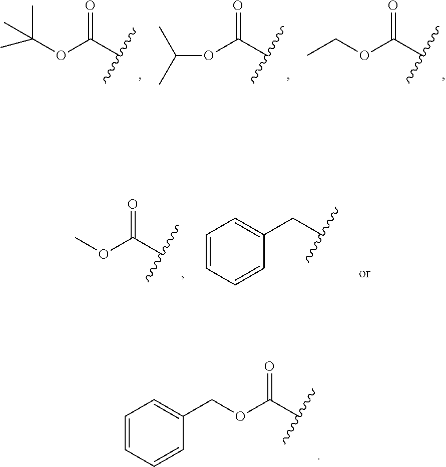

In the present invention, preferably, R₄ is H, NO₂, F, Cl, Br, I, methyl, methoxy or

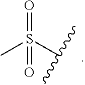 [SMILES: *S(C)(=O)=O]

In the present invention, preferably, R₃ is

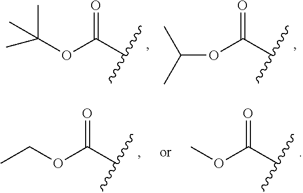

In the present invention, more preferably, R₁ is H, R₂ is

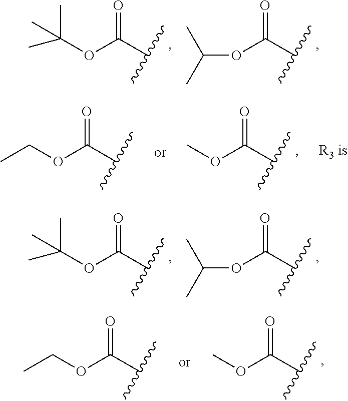

### and R₄ is F, Cl or Br.

In the present invention, preferably, the compound as shown in Formula I is

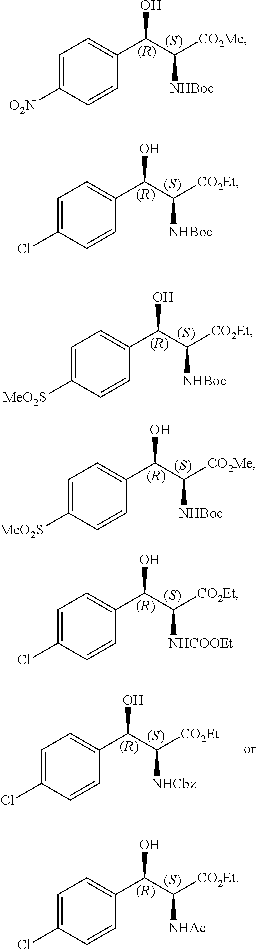

In the present invention, preferably, the compound as shown in Formula II is

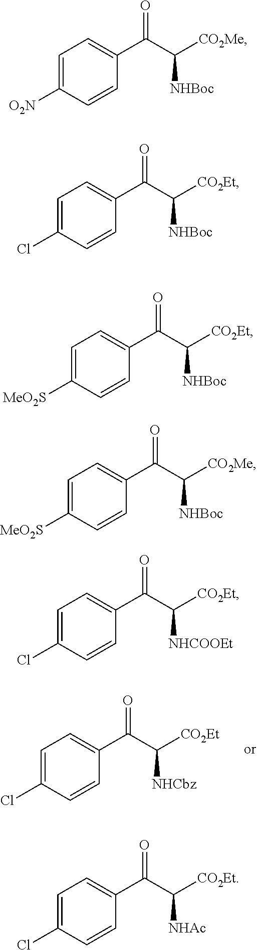

In the present invention, the coenzyme may be a conventional coenzyme in the art, preferably a reducing coenzyme and/or an oxidative coenzyme. The preferred oxidative coenzyme is NAD⁺ and/or NADP⁺; the preferred reducing coenzyme is NADH and/or NADPH.

In the present invention, the amount used of the coenzyme may be the conventional amount used of the coenzyme in the art. Preferably, the mass ratio of the coenzyme to the compound shown in Formula I is 1: (1-100), preferably 1: (50-100), for example, 1:100 or 1:75.

In the present invention, the liquid reaction system may be a conventional liquid reaction system suitable for carbonyl reductase reactions in the art, preferably comprising an enzyme for coenzyme regeneration and a co-substrate for coenzyme regeneration. The enzyme used for coenzyme regeneration is preferably one or more selected from alcohol dehydrogenase, formate dehydrogenase, and glucose dehydrogenase, such as glucose dehydrogenase. The co-substrate is preferably one or more selected from isopropanol, glucose, and ammonium formate, such as glucose.

In the present invention, the amount of co-substrate in the liquid reaction system may be a conventional amount used in the art, preferably the mass concentration of the co-substrate in the liquid reaction system is 5-30%, more preferably 5-20%, for example, 16%, 8%, or 12%.

In the present invention, the amount of enzyme used for coenzyme regeneration in the liquid reaction system may be a conventional amount in the art, preferably the mass concentration of the enzyme used for coenzyme regeneration in the liquid reaction system is 1-10%, more preferably 1%-5%, for example, 2.5%, 1.6%, or 2.3%.

In the present invention, the reaction temperature for the reduction reaction may be a conventional reaction temperature in the art, preferably 10° C.-50° C., more preferably 25° C.-35° C., for example, 30° C.

In the present invention, the reaction time of the reduction reaction is related to the reaction temperature and reaction scale, preferably 0.1-72 hours, and more preferably 3-24 hours.

In the present invention, the pH of the reduction reaction may be a conventional pH in the art, preferably 6-10, more preferably 7.0-9.0, such as 7.5-8.0.

In the present invention, the carbonyl reductase mutant is added to the reduction reaction in a conventional form in the art, preferably in the form of free enzyme, immobilized enzyme, bacterial powder or bacterial form enzyme, and more preferably in bacterial form enzyme.

In the present invention, the liquid reaction system further comprises a buffer solution, such as a phosphate buffer solution. The preferred phosphate buffer is 0.1M phosphate buffer. The phosphate buffer is used to regulate the pH of the liquid reaction system.

In the present invention, the liquid reaction system further comprises a cosolvent. The cosolvent may be a conventional cosolvent in the art; preferably selected from one or more of dimethyl sulfoxide, isopropanol, and methylbenzene, preferably dimethyl sulfoxide.

In the present invention, the amount used of the cosolvent may be a conventional amount used in the art, preferably the mass concentration of the cosolvent in the liquid reaction system is 10%-50%, more preferably 20%-30%, for example, 30%, 29%, or 28%.

In the present invention, after the reduction reaction is completed, a post-processing step is further incorporated.

In the present invention, the post-processing step is a conventional post-processing step in the art. Preferably, the post-processing step comprises: adding an organic solvent to the liquid reaction system, heating, filtering the bacterial body, extracting, washing the organic phase with water, drying, and filtering to concentrate the organic layer to obtain a compound shown in Formula II.

In the present invention, the organic solvent may be a conventional organic solvent in the art, preferably an ester solvent, an ether solvent, an alcohol solvent, an aromatic solvent, or a chlorinated alkane solvent. The preferred ester solvent is ethyl acetate or isopropyl acetate. The preferred ether solvent is methyl tert-butyl ether or 2-methyltetrahydrofuran. The preferred alcohol solvent is n-butanol. The preferred aromatic solvent is methylbenzene. The preferred chlorinated alkane solvent is dichloromethane.

In the present invention, the heating temperature is set to denature the protein, preferably at 60° C.

In the present invention, the heating time is set to denature the protein, preferably for 1 hour.

In the present invention, the water used for washing comprises pure water and water containing inorganic salts, for example, pure water and/or 5% saline solution.

In the present invention, the drying method may be a conventional drying method in the art, preferably using desiccant for drying. The preferred desiccant is anhydrous sodium sulfate.

More preferably, the post-processing step comprises: adding methyl tert-butyl ether or ethyl acetate to the reaction solution of the reduction reaction, heating, filtering the bacterial body, and extracting, washing the organic phase with pure water and 5% saline solution, drying with anhydrous sodium sulfate, and filtering to concentrate the organic layer to obtain the compound shown in Formula II.

The present invention also provides an application of the carbonyl reductase mutant in reducing carbonyl as described above.

In the present invention, preferably, the reaction substrate and reaction conditions for the application are as described in the preparation method of the compound shown in Formula II.

### Terms

Enantiomeric excess (ee): typically used to characterize the excess value of one enantiomer relative to another in chiral molecules.

Diastereomeric excess (de): typically used to characterize the excess value of one diastereomer relative to another in a molecule with two or more chiral centers.

Isomeric content (ic): typically used to characterize the percentage of one isomer to the total amount of all isomers in a molecule with two or more chiral centers.

### (R, S)-Carbonyl Reductase

In the present invention, “stereoselective carbonyl reductase” refers to an enzyme capable of stereoselective asymmetric catalytic reduction of prochiral ketones to chiral alcohols.

Typically, in the present invention, the stereoselective carbonyl reductase is preferably (R, S)-carbonyl reductase, with stereoselectivity defined as enantiomeric excess (ee)≥80% and diastereomeric excess (de)≥80%.

Similarly, when (R, R)-carbonyl reductase is used, stereoselectivity is defined as enantiomeric excess (ee)≥80%, diastereomeric excess (de)≥80%, and so on.

### Coenzyme

In the present invention, “coenzyme” refers to a coenzyme that can achieve electron transfer in redox reactions.

### Cosolvent

In the present invention, cosolvent can be added or not added to the reaction system.

As used herein, the term “cosolvent” refers to a third substance which is added to form soluble intermolecular complex, association, or complex salts with insoluble substances in the solvent, in order to increase the solubility of insoluble substances in the solvent. This third substance is called a cosolvent.

### Principle of Dynamic Reduction Kinetic Separation Reaction

When a single-configuration prochiral ketone (such as R-configuration) is stereoselectively reduced by carbonyl reductase, prochiral ketone with another configuration (such as S-configuration) achieves racemization of the α-chiral configuration through the enol tautomerism of the carbonyl group. Reduction and racemization are carried out under the same reaction condition, and the purpose of efficiently constructing secondary alcohols with two chiral centers is achieved.

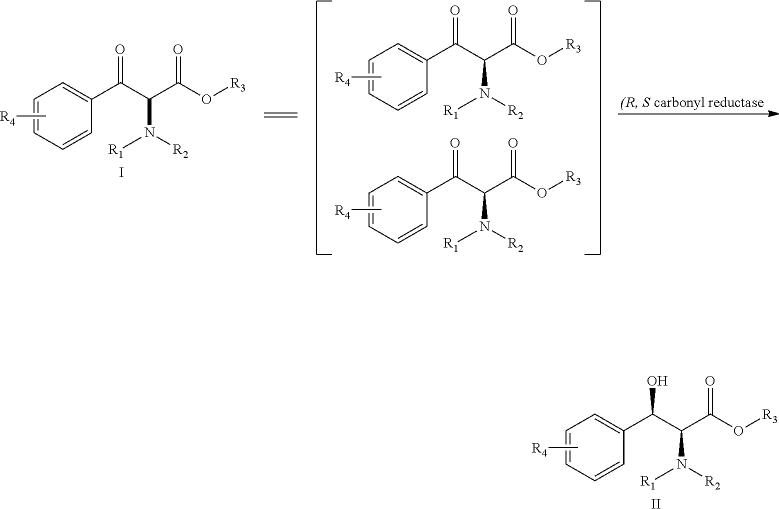

Typically, in the present invention, carbonyl reductases that only recognize I-S(S-configuration Compound I) are obtained through screening, and chiral hydroxyl groups are obtained by stereoselective reduction of carbonyl groups. Unrecognized I-R (R-configuration Compound I) is racemized and converted to I-S. The racemization and reduction processes are connected and promoted, and theoretically, the desired chiral products can be obtained 100%. The reduction of prochiral carbonyl substrates through carbonyl reductase and the enol tautomerization and racemization are cleverly combined to construct two chiral centers efficiently and economically in one step, which shows promising application and development prospects.

When not contrary to common knowledge in the field, the preferred conditions mentioned above can be arbitrarily combined to obtain the preferred examples of the present invention.

The reagents and raw materials used in the invention are commercially available.

The positive progressive effects of the invention are as follows:

The carbonyl reductase mutant of this invention has higher enzyme activity than the wild-type carbonyl reductase. The activity of some carbonyl reductase mutants is 50 times that of wild-type carbonyl. The carbonyl reductase mutant of this invention can be applied to catalyze the carbonyl reduction reaction of the compound shown in Formula I.

The preparation method provided by this invention can prepare compounds shown in Formula II with conversion rate >99%, chirality ee value >99% and chirality de value >99%. The preparation method of the invention has the characteristics of green, environmental friendly and economy.

### BRIEF DESCRIPTION OF THE DRAWINGS

FIG. 1 shows a liquid chromatography comparison of the chiral purity of the four isomers of racemic Compound 1b in comparative example 1 and the Compound 1b prepared by WTEA enzyme conversion reaction.

FIG. 2 shows a liquid chromatography comparison of the chiral purity of the four isomers of racemic Compound 1b in comparative example 1 and the Compound 1b prepared by the mutant carbonyl reductase (Mutant 65) conversion reaction.

FIG. 3 shows the liquid phase diagram of the four isomers of racemic Compound 2b in comparative example 1.

FIG. 4 shows the liquid phase diagram of the chiral purity of Compound 2b prepared by the WTEA enzyme conversion reaction in comparative example 1.

FIG. 5 shows the liquid phase diagram of the chiral purity of Compound 2b prepared by the mutant carbonyl reductase (Mutant 65) conversion reaction in comparative example 1.

FIG. 6 shows the liquid phase diagram of the four isomers of racemic Compound 5b in comparative example 2.

FIG. 7 shows the liquid phase diagram of the chiral purity of Compound 5b prepared by the WTEA enzyme conversion reaction in comparative example 2.

FIG. 8 shows the liquid phase diagram of the chiral purity of Compound 5b prepared by the mutant carbonyl reductase (Mutant 65) conversion reaction in comparative example 2.

### DETAILED DESCRIPTION

The present invention will be further explained through embodiments, but will not be limited within the scope of the embodiments. The experimental methods in the following embodiments without specific conditions shall be selected according to conventional methods and conditions, or according to the product manual. The experimental methods in the following embodiments without specific conditions are usually carried out under conventional conditions such as those described in Sambrook et al., Molecular Cloning: Laboratory Manual (New York: Cold Spring Harbor Laboratory Press, 1989), or as recommended by the manufacturer. Unless otherwise specified, percentages and portions are calculated by weight.

Specifically, the biological preparation method of the present invention uses Compound I (such as Compound 1) as the raw material, and carbonyl reductase as the biocatalyst, efficiently prepares Compound II with a stereoconformation (reduction yield>99%, chiral ee value >99%, chiral de value >99%) in the presence of coenzyme, and constructs two chiral centers through a one-step reaction, greatly improving production efficiency and reducing production costs.

### Material

The gene synthesis was completed by Nanjing GenScript.

The coding genes were obtained through commercial gene synthesis, and then constructed into an expression vector and introduced into the host bacteria to induce expression of carbonyl reductase and obtain it.

The preparation of substrate for enzyme reduction, Compound I, can be found in the method described in Tetrahedron 2016, 72:1787-1793

### Method

By using conventional techniques in this field, the glucose dehydrogenase and formate dehydrogenase used for achieving coenzyme regeneration mentioned above, and target genes and their mutants were respectively constructed into pET28a (+) vectors, and then introduced into the expression host Escherichia coli. By inducing expression, bacterial cells containing glucose dehydrogenase, formate dehydrogenase, and carbonyl reductase were obtained respectively. Subsequent biological conversion reactions can be performed using the bacterial cells obtained directly by centrifugation, or the crude enzyme solution or crude enzyme powder obtained by breaking the wall of the bacterial cells.

### 2. Method for Preparing Compound II by Biocatalytic Reduction of Compound I

The present invention provides a method for preparing compound II through catalytic reduction of Compound I by carbonyl reductase. The reaction formula is as follow:

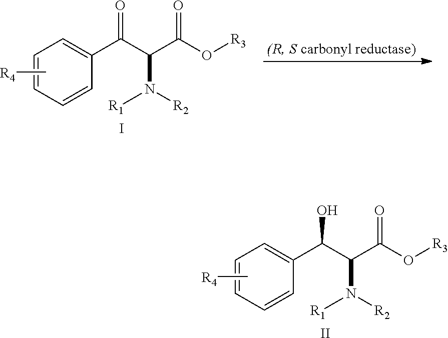

R₁ is H,

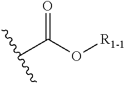

or benzyl; or benzyl; R₁₋₁ is C₁-C₆ alkyl or benzyl; R₂ is H,

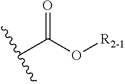

or benzyl;
   R₂₋₁ is C₁-C₆ alkyl or benzyl; R₃ is

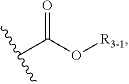

wherein R₃₋₁ is C₁-C₆ alkyl;
R₄ is H, NO₂, halogens, C₁-C₆ alkyl, C₁-C₆ alkoxy, or C₁-C₆ alkyl substituted sulfonyl.

Among them, the biocatalytic system includes carbonyl reductase and coenzyme. In the present invention, the nucleotide sequence of the gene encoding the carbonyl reductase is as shown in SEQ ID NO: 2, and the amino acid sequence of the carbonyl reductase is as show in SEQ ID NO: 1. According to common knowledge in this field, the above-mentioned gene of carbonyl reductase can be obtained through commercial gene synthesis.

According to the above preferred system, the process of the preparation method is as follows: the substrate is fully dissolved in a cosolvent, such as dimethyl sulfoxide or isopropanol, then added into a phosphate buffer and stirred evenly. The solution is added with bacterial cells, crude enzyme solution, crude enzyme powder or pure enzyme, then added with coenzyme NADP+ and co-substrate glucose, maintained at 20° C. to 40° C. and monitored by TLC or HPLC. The reaction is terminated when the remaining raw material is less than 2%. The reaction solution is extracted using organic solvents, which can be methyl tert-butyl ether, toluene, ethyl acetate, isopropyl acetate, dichloromethane, 2-methyltetrahydrofuran, or n-butanol. The water layer is extracted 2-3 times. The organic phases are merged; washed 2-3 times with saturated saline and concentrated to obtain a light yellow solid, which is Compound II (1b-7b depending on the substrate, as shown in Table 3).

The final concentration of substrate Compound I in the system is 10-200 g/L, the reaction temperature is 20-40° C., the rotational speed is 200 rpm/min, and the reaction time is about 3-24 hours. The reaction time varies depending on the substrate concentration or the monitoring of raw material conversion by HPLC. Generally, the reaction is terminated when the remaining raw material is less than 2%.

### 3. Positive Chirality Monitoring Method for Compound II:

The sample is dissolved in methanol at a concentration of 10 mg/mL; injection volume is 2 μL. The specific detection methods are shown in Table 2.

TABLE 2
Positive chirality monitoring method ^{a}.

| | | | Measure | |
|---|---|---|---|---|
| | | | wavelength | |
| Compound | Column | Mobile phase | (nm) | Retention time (min) |
| 1b | IB-3^{b} | n-hexane:isopropanol = 95:5 | 254 | 22.0 min, 26.8 min, 38.7 min, 46.8 min |
| 2b | OJ-H^{c} | n-hexane:isopropanol = 95:5 | 210 | 8.3 min, 10.3 min, 11.6 min, 15.8 min |
| 3b | IB-3 | n-hexane:ethanol = 95:5, (0.1% diethylamine, 0.1% trifluoroacetic acid) | 220 | 32.2 min, 34.3 min, 37.9 min, 39.1 min |
| 4b | IB-3 | n-hexane:ethanol = 95:5, (0.1% diethylamine, 0.1% trifluoroacetic acid) | 220 | 41.6 min, 44.2 min, 46.8 min, 50.3 min |
| 5b | OJ-H | n-hexane:isopropanol = 93:7 | 210 | 12.3 min, 14.7 min, 17.3 min, 30.9 min |
| 6b | OJ-H | n-hexane:isopropanol = 32:68 | 210 | 5.4 min, 5.7 min, 6.3 min, 7.2 min |
| 7b | OJ-H | n-hexane:isopropanol = 93:7 | 210 | 16.1 min, 19.4 min, 21.9 min, 23.6 min |

^{a} General testing conditions: temperature: 30° C.; flow rate: 1 mL/min
^{b}IB-3: CHIRALPAK IB-3 column (3 μm, 4.6 mm × 250 mm, DAICEL, Shanghai)
^{c}OJ-H: CHIRALPAK IB-3 column (5 μm, 4.6 mm × 250 mm, DAICEL, Shanghai)

TABLE 3
Structure of 1b-7b compounds

| Compound | Compound structure |
|---|---|
| 1b | 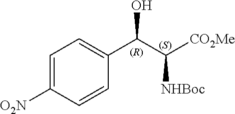 |
| 2b | 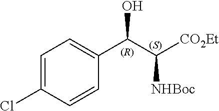 |
| 3b | 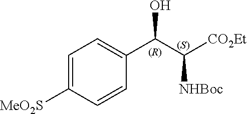 |
| 4b | 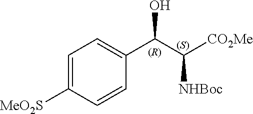 |
| 5b | 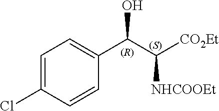 |
| 6b | 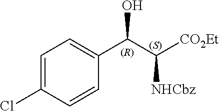 |
| 7b | 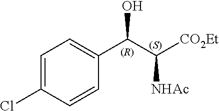 |

### 4. Reverse Phase Monitoring Method for Compound II:

HPLC conditions: phenomenex Gemini 5u C18 110A, 250×4.6 mm, 5 μm; flow rate: 1 mL/min; mobile phase: acetonitrile:water=55:45; UV detection wavelength: 210, 220, 245 nm; column temperature: 30° C.; sample concentration: 10 mg/mL; injection volume 10 μL.

### Example 1: Construction of Genetically Engineered Bacteria Expressing Recombinant Carbonyl Reductase and Glucose Dehydrogenase

The target genes of carbonyl reductase WTEA (nucleotide sequence SEQ ID NO: 2, amino acid sequence SEQ ID NO: 1) and glucose dehydrogenase GDH were commissioned to a commercial company for gene synthesis. They were separately cloned into pET28a (+) vector and transferred into Escherichia coli DH5a sensory cells, cultured on agar plates, and positive transformant colonies were selected and plasmids were extracted for sequencing. Recombinant plasmids were extracted and introduced into the BL21 (DE3) strain. Single bacteria were selected and cultured in LB, and the genetically engineered bacteria pET28a(+)-WTEA which can be induced to express recombinant carbonyl reductase and the genetically engineered bacteria pET28a(+)-GDH which expresses recombinant glucose dehydrogenase were obtained.

### Example 2: Preparation of Recombinant Carbonyl Reductase and Glucose Dehydrogenase

The genetically engineered bacteria previously stored in glycerol were inoculated to a LB liquid medium containing 50 μg/mL kanamycin, incubated at 37° C., 220 rpm, for 14 hours to obtain seed culture medium. The seed culture medium were inoculated at a ratio of 1.5% to a LB liquid medium with 50 μg/mL kanamycin resistance, cultured at 37° C. and 220 rmp until the OD₆₀₀ value >2.0. Isopropylthiogalactoside (IPTG) was added at a final concentration of 1 mM The solution was cooled to 25° C. to induce protein expression, cultured continuously for 20 hours, placed in a jar, and centrifuged (centrifuge at 4000 g for 30 minutes) to obtain bacterial cells for preparation of bioconversion.

LB liquid culture medium (g/L): tryptone 10.0, yeast extract 5.0, NaCl 10.0, 1 L deionized water, pH 7.0.

Fermentation medium 2 (g/L): yeast extract 24.0, soy peptone 12.0, NaCl 3.0, glycerol 5.0, K₂HPO₄·3H₂O 2.0, MgSO₄·7H₂O 0.5, deionized water 1 L, pH 7.5.

### Example 3: Construction and Screening of Carbonyl Reductase WTEA Mutants

The wild-type carbonyl reductase gene WTEA was mutated by directed evolution to obtain a plasmid library containing the evolved carbonyl reductase genes. Then they were transferred into Escherichia coli BL21 (DE3) (product number: Kangwei Century CW0809S). The bacteria were plated on LB solid culture medium with 50 μg/mL kanamycin. After being cultured in a 37° C. oven for 14 hours, the single colony was selected and transferred to a 96 well plate containing 400 μL LB liquid culture medium (containing 50 μg/mL kanamycin, incubated overnight at 37° C. and 200 rpm to obtain the seed solution. Then 10 μL seed liquid was transferred to a 96 deep-well plate containing 400 μL fermentation medium (fermentation medium 2, containing 50 μg/mL kanamycin), cultivated at 37° C. and 200 rpm for 3 hours. Then isopropylthiogalactoside (IPTG) was added at a final concentration of 1 mM. The solution was cooled to 25° C. to induce mutant expression, and cultured continuously for 20-24 hours. The cells were then centrifuged at 4000 g, 30 min for precipitation, and re-suspended in 200 μL lysate buffer (0.1 M phosphate buffer containing 1000 U lysozyme, pH 7.0) for lysis at 30° C. for 1 h. Then the 96 deep-well plate was centrifuged at 4° C. at 4000 g for 30 min, and the clarified supernatant was used to determine the activity of the mutants. 190 μL reaction solution (containing 0.4 mM substrate, 1 mM NADPH, 40 μL dimethyl sulfoxide) was added to a new 96-well plate, and then 10 μL supernatant was added. The change of NADPH was detected at 340 nm. The consumption of NADPH reflects the level of enzyme activity of the mutants, and the relative activity of each mutant is shown in Table 4.

TABLE 4
Mutants and their relative activity

| Serial | | | |
|---|---|---|---|
| number | | | Relative |
| of | Amino acid residue difference | Number of | activity |
| mutant | from SEQ ID NO:1 | mutation | (%)* |
| 5 | A190V, S193A | 2 | 364 |
| 7 | R142F, A190V, S193A | 3 | 402 |
| 9 | R142M, A190V, S193A | 3 | 428 |
| 11 | A138V, R142F, A190V, S193A | 4 | 196 |
| 13 | R142L, A190V, S193A | 3 | 342 |
| 15 | A138V, R142L, A190V, S193A | 4 | 373 |
| 17 | A138L, R142L, A190V, S193A | 4 | 405 |
| 19 | F88V, R142M, A190V, S193A | 4 | 1037 |
| 21 | F88I, R142L, A190V, S193A | 4 | 946 |
| 23 | F88I, R142M, A190V, S193A | 4 | 627 |
| 25 | F88I, R142F, A190V, S193A | 4 | 801 |
| 27 | F88V, R142L, A190V, S193A | 4 | 1398 |
| 29 | F88S, R142H, A190V, S193A | 4 | 1419 |
| 31 | F88V, A138V, R142L, A190V, | 5 | 1152 |
| | S193A | | |
| 33 | F88I, A138L, R142I, A190V, S193A | 5 | 1028 |
| 35 | F88I, A138L, R142F, A190V, S193A | 5 | 1311 |
| 37 | F88I, A138L, R142L, A190V, S193A | 5 | 1214 |
| 39 | F88V, A138L, R142L, A190V, S193A | 5 | 1884 |
| 41 | F88I, A138L, R142M, A190V, S193A | 5 | 2160 |
| 43 | F88V, A138L, R142M, A190V, S193A | 5 | 2280 |
| 45 | W82L, F88V, A138L, R142M, A190V, | 6 | 2861 |
| | S193A | | |
| 47 | W82L, V121A, A138L, A190V, S193A, | 6 | 467 |
| | K206H | | |
| 49 | W82L, F88V, V121A, A138L, R142M, | 7 | 4051 |
| | A190V, S193A | | |
| 51 | W82L, F88V, V121A, A138L, R142M, | 8 | 4562 |
| | A190V, S193A, Y201F | | |
| 53 | W82L, F88V, V121A, A138L, R142M, | 9 | 5109 |
| | A190V, S193A, N204A, K206H | | |
| 55 | W82L, F88V, V121A, A138L, R142M, | 9 | 5024 |
| | A190V, S193A, N204G, K206H | | |
| 57 | W82L, F88V, V121A, A138L, R142M, | 9 | 5223 |
| | A190V, S193A, K206H, K207N | | |
| 59 | W82L, F88I, V121A, A138L, R142M, | 9 | 4668 |
| | A190V, S193A, K206H, K207N | | |
| 61 | W82L, F88I, V121A, A138L, R142M, | 10 | 5128 |
| | A190V, S193A, Y201F, K206H, K207N | | |
| 63 | W82L, F88V, V121A, A138L, R142M, | 10 | 5346 |
| | A190V, R192M, S193A, N204A, K206H, | | |
| 65 | W82L, F88V, V121A, A138L, R142M, | 9 | 5415 |
| | A190V, S193A, Y201F, N204A | | |

*The activity of wild-type carbonyl reductase gene WTEA (SEQ ID NO: 1) was set to 100%.

### Example 4: Biocatalytic Preparation of Compound 1b

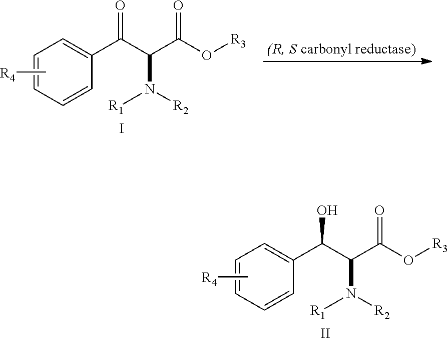

Wherein R₁ is H; R₂ is Boc; R₃ is CH₃; R₄ is NO₂, I is Compound 1a, and II is Compound 1b. 0.1M phosphate buffer (140 mL) was added with glucose (40 g), NADP⁺ (0.2 g), the bacteria cells of Mutant 65 (20 g) obtained by the fermentation mentioned above, and bacteria cells of glucose dehydrogenase (GDH) (6 g), vigorously stirred and the DMSO (60 mL) solution of Compound 1a (20 g) was slowly added at 30° C. with an interval of 0.5 h. The pH was adjusted to 7.5-8.0 by 5% sodium carbonate aqueous solution, and the reaction was terminated when the reaction conversion rate was >98% monitored by HPLC.

Methyl tertiary ether (200 mL) was added for extraction. The solution was heated to 60° C. for 1 h to inactivate the protein in the bacteria. 10% diatomaceous earth was added to filter the bacteria. The water layer was extracted with methyl tertiary ether (100 mL). The organic layers were combined, washed by water (100 mL×2) and then by 5% saline, dried by anhydrous sodium sulfate, filtered, and then concentrated to obtain light yellow oil which is Compound 1b (17.6 g) with ee value of 99.9% and de value of 99.9%.

### Example 5: Biocatalytic Preparation of Compound 2b

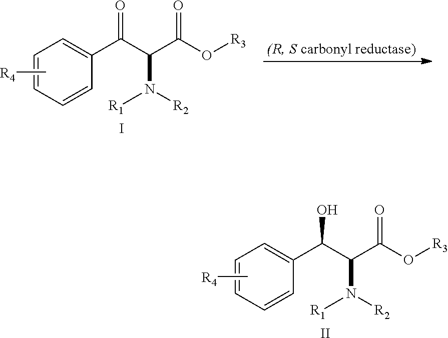

Wherein R₁ is H; R₂ is Boc; R₃ is CH₂CH₃; R₄ is Cl, I is Compound 2a, and II is Compound 2b.

0.1M phosphate buffer (70 mL) was added with glucose (10 g), NADP⁺ (0.1 g), the bacteria cells of Mutant 65 (10 g) obtained by the fermentation mentioned above, and bacteria cells of glucose dehydrogenase (GDH) (3 g), vigorously stirred and the DMSO (30 mL) solution of Compound 2a (7.5 g) was added in batches at 30° C. with an interval of 0.5 h. The pH was adjusted to 7.5-8.0 by 5% sodium carbonate aqueous solution. The reaction was terminated when the reaction conversion rate was >98% monitored by HPLC.

Methyl tertiary ether (100 mL) was added for extraction. The solution was heated to 60° C. for 1 h to inactivate the protein in the bacteria. 10% diatomite was added to filter the bacteria. The water layer was extracted with methyl tertiary ether (50 mL). The organic layers were combined, washed by water (50 mL×2) and then by 5% saline, dried by anhydrous sodium sulfate, filtered and concentrated to obtain light yellow oil substance which is Compound 2b (6.6 g) with ee value of 99.9% and de value of 99.9%.

### Example 6: Biocatalytic Preparation of Compound 3b

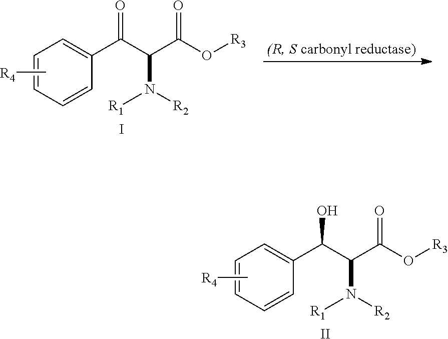

Wherein R₁ is H; R₂ is Boc; R₃ is CH₂CH₃; R₄ is SO₂Et, I is Compound 3a, and II is Compound 3b.

0.1M phosphate buffer (70 mL) was added with glucose (15 g), NADP⁺ (0.1 g), the bacteria cells of Mutant 65 (10 g) obtained by the fermentation mentioned above, and bacteria cells of glucose dehydrogenase (GDH) (2 g), vigorously stirred and the DMSO (30 mL) solution of Compound 3a (10 g) was added in batches at 30° C. with an interval of 0.5 h. The pH was adjusted to 7.5-8.0 by 5% sodium carbonate aqueous solution. The reaction was terminated when the reaction conversion rate was >98% monitored by HPLC.

Methyl tertiary ether (200 mL) was added for extraction. The solution was heated to 60° C. for 1 h to inactivate the protein in the bacteria. 10% diatomite was added to filter the bacteria. The water layer was extracted with methyl tertiary ether (100 mL). The organic layers were combined, washed by water (100 mL×2) and then by 5% saline, dried by anhydrous sodium sulfate, filtered and concentrated to obtain light yellow oil substance which is Compound 3b (4.7 g) with ee value of 99.9% and de value of 99.9%.

### Example 7: Biocatalytic Preparation of Compound 4b

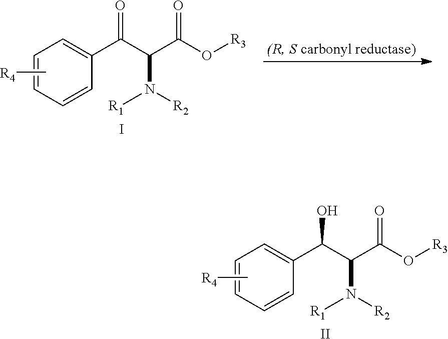

Wherein R₁ is H; R₂ is Boc; R₃ is CH₂CH₃; R₄ is SO₂Me, I is Compound 4a, and II is Compound 4b.

0.1 m phosphate buffer (140 ml) was added with glucose (40 g), NADP⁺ (0.2 g), the bacteria cells of Mutant 65 (20 g) obtained by the fermentation mentioned above, and bacteria cells of glucose dehydrogenase (GDH) (6 g), vigorously stirred and the DMSO (60 mL) solution of Compound 4a (20 g) was added in batches at 30° C. with an interval of 0.5 h. The pH was adjusted to 7.5-8.0 by 5% sodium carbonate aqueous solution. The reaction was terminated when the reaction conversion rate was >98% monitored by HPLC.

Methyl tertiary ether (200 mL) was added for extraction. The solution was heated to 60° C. for 1 h to inactivate the protein in the bacteria. 10% diatomite was added to filter the bacteria. The water layer was extracted with methyl tertiary ether (100 mL). The organic layers were combined, washed by water (100 mL×2) and then by 5% saline, dried by anhydrous sodium sulfate, filtered, concentrated to obtain light yellow oil substance which is Compound 4b (17.6 g) with ee value of 99.9% and de value of 99.9%.

### Comparative Example 1: Comparison of Compounds 1b, 2b, 4b Prepared by Biocatalytic of Wild-Type Carbonyl Reductase and Mutant Carbonyl Reductase

140 mL of 0.1M phosphate buffer was added with 40 g of glucose, 0.2 g of NADP⁺, 6 g of glucose dehydrogenase (GDH), and 20 g of fermented bacteria cells of wild-type carbonyl reductase (or Mutant 47, or Mutant 63, or Mutant 65), violently stirred, and DMSO (60 mL) solution containing 20 g substrate 1a (or 20 g substrate 2a, 20 g substrate 4a) was slowly added at 30° C. with an interval of 0.5 h. The pH was adjusted to 7.5-8.0 by 5% sodium carbonate aqueous solution. The reaction was terminated after 24 h, and the conversion rate was monitored by HPLC.

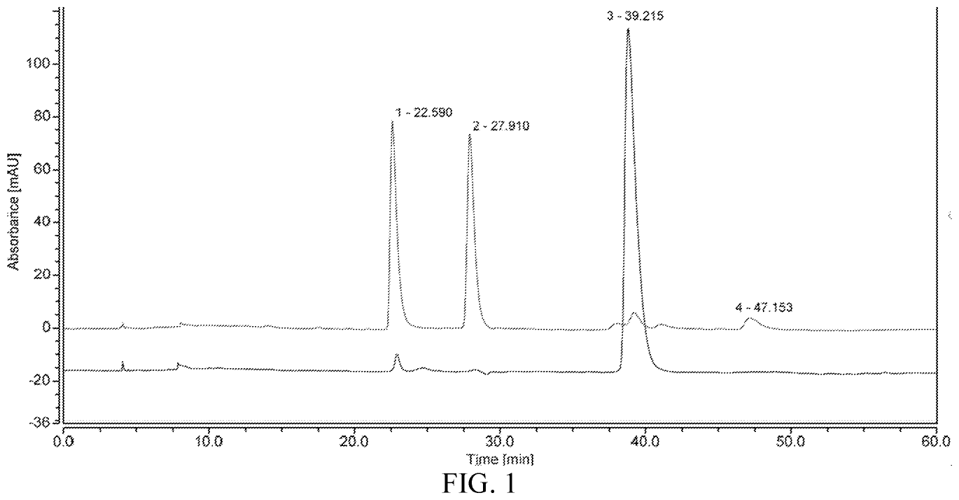

The liquid chromatography comparison of the four isomers of racemic Compound 1 and the chiral purity of Compound 1b prepared by WTEA enzymatic conversion reaction is shown in FIG. 1. The retention time of the four isomers is 23 min; 28 min; 39 min; and 47 min, respectively. The retention time of Compound 1b prepared by WTEA enzymatic conversion reaction is 39 min, and the chiral purity (de) is 98%.

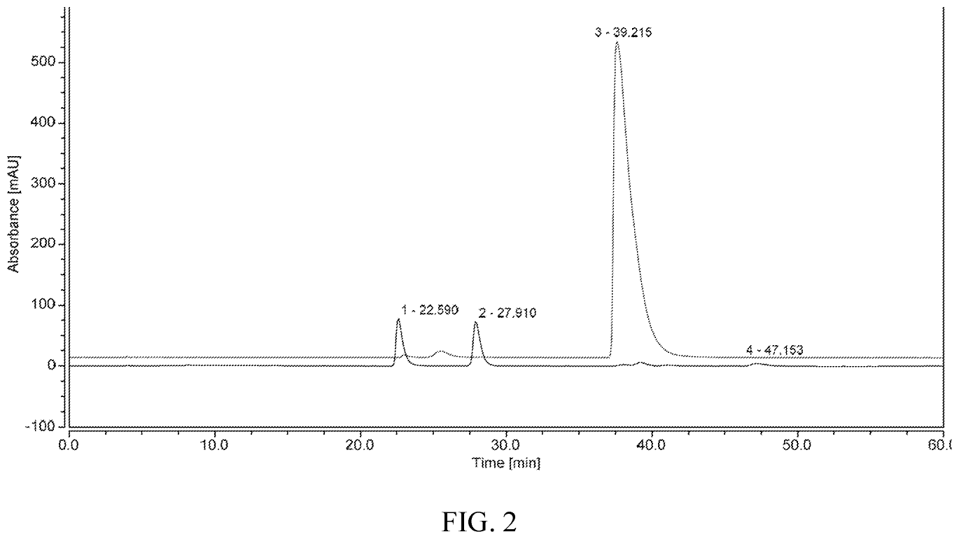

The liquid chromatography comparison of the four isomers of racemic Compound 1b and the chiral purity of Compound 1b prepared by the conversion reaction using the mutant carbonyl reductase (Mutant 65) is shown in FIG. 2. The retention time of Compound 1b obtained by the conversion reaction using Mutant 65 is 39 min, and the chiral purity (de) is 99%.

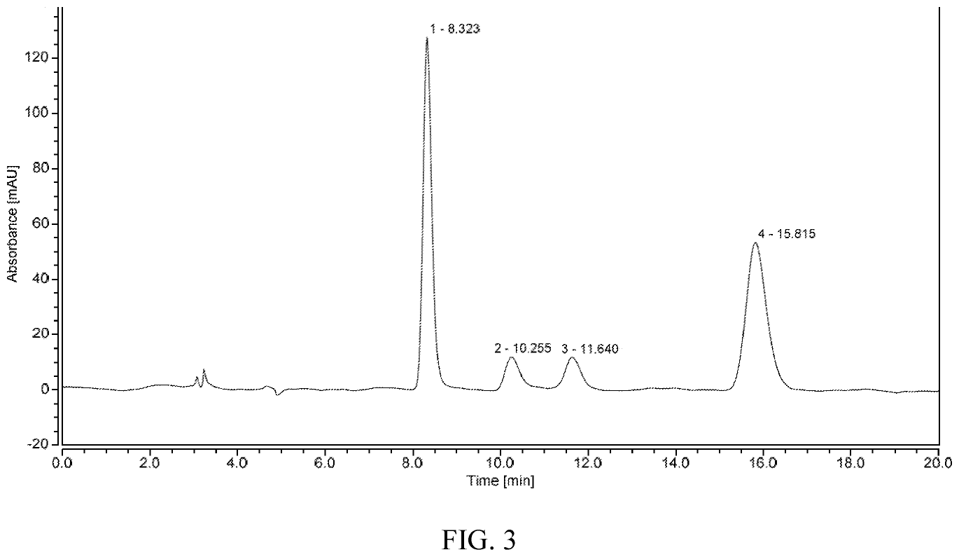

The liquid phase diagrams of the four isomers of racemate Compound 2b are shown in FIG. 3, and the retention time of the four isomers is 8 min; 10 min; 12 min; 16 min, respectively.

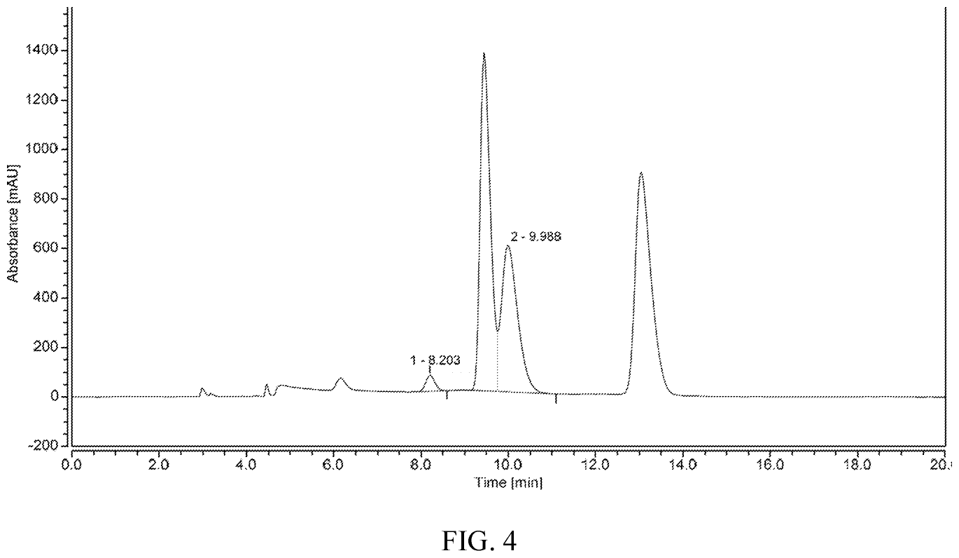

The liquid phase diagram of the chiral purity of Compound 2b prepared by WTEA enzyme conversion reaction is shown in FIG. 4. The retention time of Compound 2b obtained by WTEA enzyme conversion reaction is 10 min, and the chiral purity (de) is 89%.

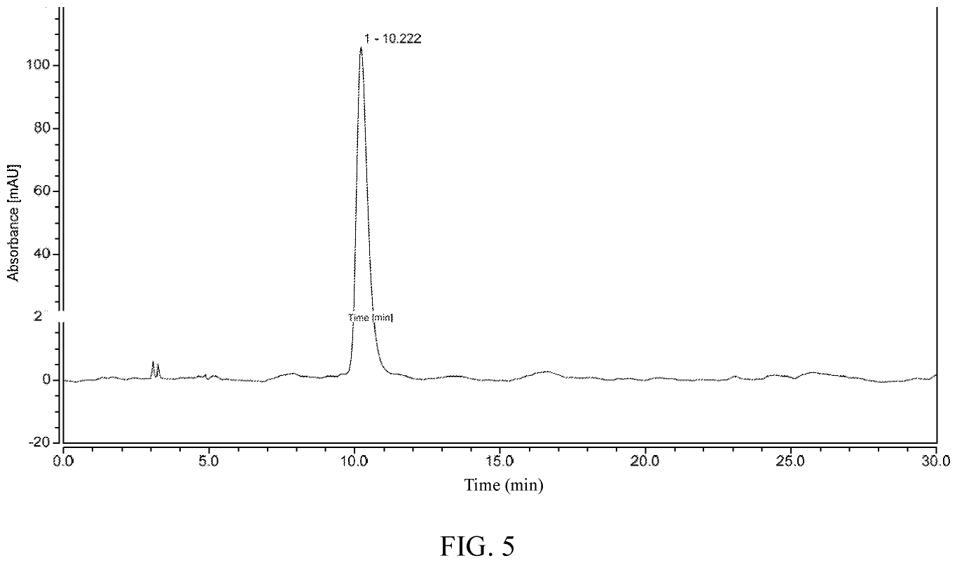

The liquid phase diagram of the chiral purity of Compound 2b prepared by the conversion reaction using mutant carbonyl reductase (Mutant 65) is shown in FIG. 5. The retention time of Compound 2b obtained by the conversion reaction of Mutant 65 is 10 min, and the chiral purity (de) is 99%.

TABLE 5
Comparison of catalysis activity and stereoselectivity of wild-type
carbonyl reductase and mutant carbonyl reductase (1a, 2a, 3a)

| | | Conversion | Stereoselectivity of |
|---|---|---|---|
| Type of enzyme | substrate | Rate (%) | product (%) |
| SEQ ID NO: 1 | 1a | 4 | ee = 99, de = 98 |
| | 2a | 6 | ee = 99, de = 89 |
| | 4a | 35 | ee = 99, de = 99 |
| Mutant 47 | 1a | 25 | ee = 99, de = 70 |
| | 2a | 22 | ee = 99, de = 89 |
| | 4a | 65 | ee = 99, de = 98 |
| Mutant 63 | 1a | 100 | ee = 99, de = 99 |
| | 2a | 98 | ee = 99, de = 99 |
| | 4a | 100 | ee = 99, de = 99 |
| Mutant 65 | 1a | 100 | ee = 99, de = 99 |
| | 2a | 99 | ee = 99, de = 99 |
| | 4a | 100 | ee = 99, de = 99 |

Note:
SEQ ID NO: 1 is a wild type carbonyl reductase. Mutant 47 is from patent application CN109207531A.

### Comparative Example 2: Comparison of Stereoselectivity of Compound 1-7b Prepared by Biocatalytic of Wild-Type Carbonyl Reductase and Mutant Carbonyl Reductase

Formulation of a 2 mL reaction system with different substrates and different carbonyl reductases: substrate 1a-7a (20 mM), 10% dimethyl sulfoxide (v/v), glucose (40 mM), NADP⁺ (0.2 mM), carbonyl reductase bacteria cell (50 g/L WTEA or Mutant 65), glucose dehydrogenase bacteria cell (25 g/L), phosphate buffer (0.1 M, pH 7.0). The reaction mixture reacted at 30° C., 220 rpm for 24 hours. After the reaction, the reaction liquid was extracted with methylene chloride, and the organic layer was dried with anhydrous sodium sulfate. Finally, the stereoselectivity of Compound 1-7b prepared by biocatalytic of wild-type carbonyl reductase and mutant carbonyl reductase method was tested by HPLC.

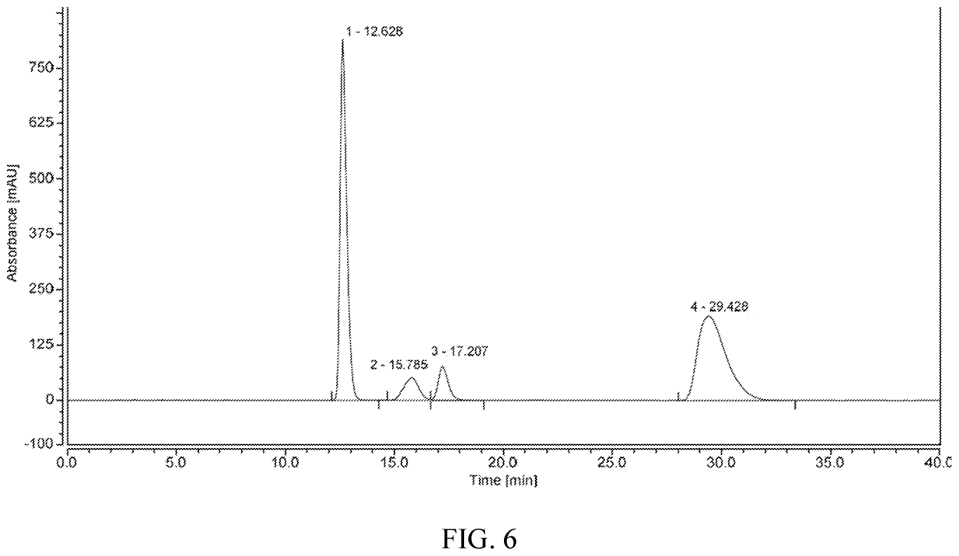

The liquid phase diagram of the four isomers of racemate Compound 5b is shown in FIG. 6, and the retention time of the four isomers is 13 min; 16 min; 17 min; 29 min, respectively.

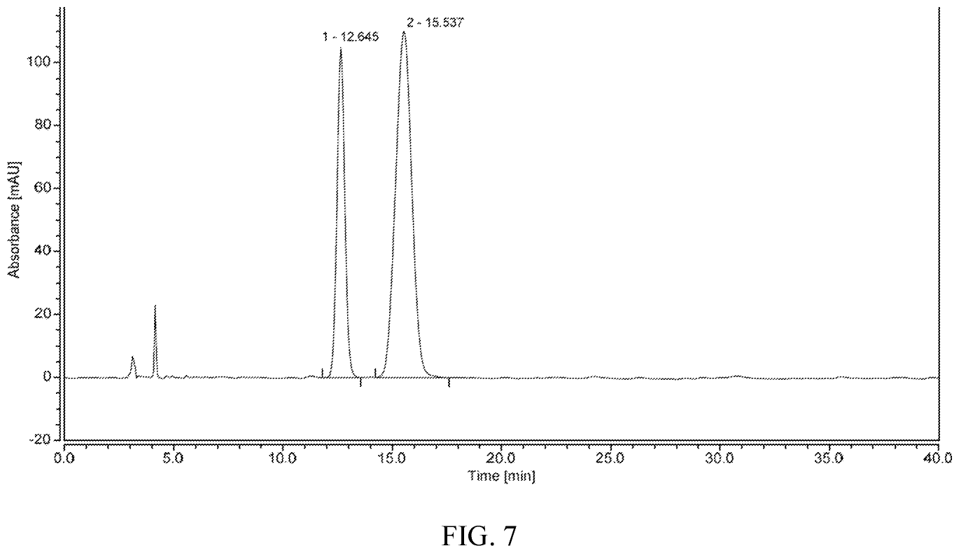

The liquid phase diagram of the chiral purity of Compound 5b prepared by WTEA enzyme conversion reaction is shown in FIG. 7. The retention time of Compound 5b obtained by WTEA enzyme conversion reaction is 16 min, and the chiral purity (ic) is 69%.

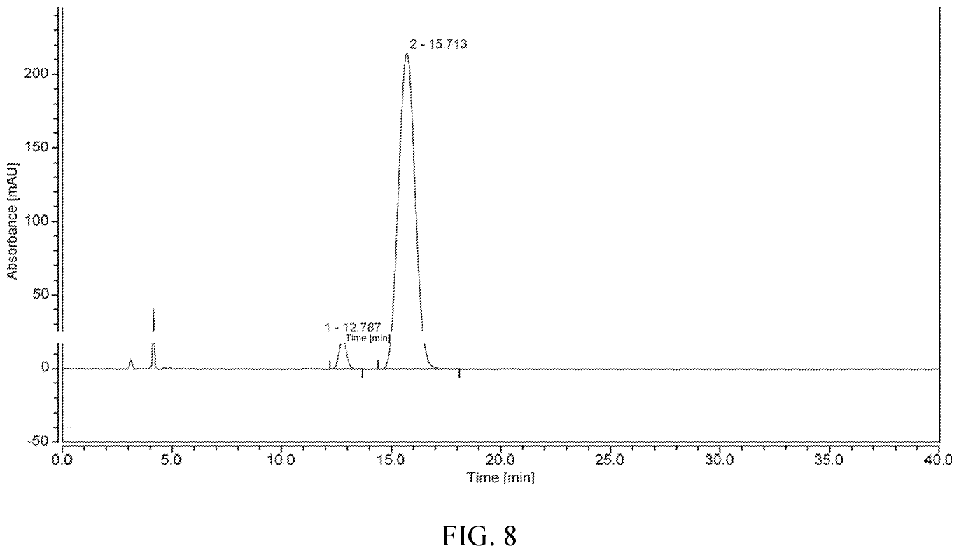

The liquid phase diagram of the chiral purity of Compound 5b prepared by the conversion reaction using mutant carbonyl reductase (Mutant 65) is shown in FIG. 8. The retention time of Compound 5b obtained by the conversion reaction using Mutant 65 is 16 min, and the chiral purity (ic) is 96%.

TABLE 6
Stereoselectivity comparison between wild-type carbonyl reductase
and mutant carbonyl reductase (Mutant 65) catalyzed
substrate (1-7a) for the preparation of Compound 1-7b

| Serial | | | ic goal |
|---|---|---|---|
| number | Product structure | Enzyme | (%) |
| 1b | 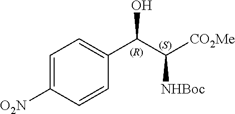 | WTEA Mutant 65 | 99 >99 |
| 2b | 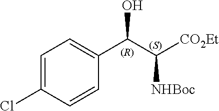 | WTEA Mutant 65 | 94 >99 |
| 3b | 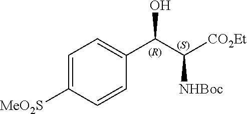 | WTEA Mutant 65 | 96 >99 |
| 4b | 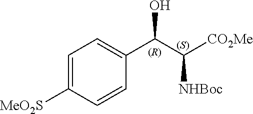 | WTEA Mutant 65 | >99 >99 |
| 5b | 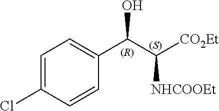 | WTEA Mutant 65 | 69 96 |
| 6b | 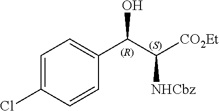 | WTEA Mutant 65 | 90 >99 |
| 7b | 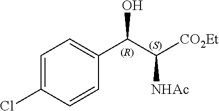 | WTEA Mutant 65 | >99 >99 |

Although the specific embodiments of the invention are described above, it should be understood by those skilled in the art that these embodiments are only examples and that a variety of changes or modifications can be made without deviating from the principle and substance of the invention. Therefore, the scope of protection of the invention is only limited by the attached claims.

1. A carbonyl reductase mutant, wherein the mutation site of the carbonyl reductase mutant comprises positions 88, 142, 190, and 193 of the amino acid sequence as shown in SEQ ID NO: 1.

2. The carbonyl reductase mutant of claim 1, wherein the mutation site of the carbonyl reductase mutant further comprises one or more positions selected from positions 82, 121, 138, 192, 201, 204, 206, and 207 of the amino acid sequence as shown in SEQ ID NO: 1;
preferably, the mutation site of the carbonyl reductase mutant further comprises at least 3 positions selected from positions 82, 121, 138, 192, 201, 204, 206, and 207 of the amino acid sequences as shown in SEQ ID NO: 1.

3. The carbonyl reductase mutant of claim 1, wherein the mutation site of the carbonyl reductase mutant is selected from any one of the following groups:
(1) the mutation sites of the carbonyl reductase mutant are positions 82, 88, 121, 138, 142, 190 and 193 of the amino acid sequence as shown in SEQ ID NO: 1;
(2) the mutation sites of the carbonyl reductase mutant are positions 82, 88, 121, 138, 142, 190, 193 and 201 of the amino acid sequence as shown in SEQ ID NO: 1;
(3) the mutation sites of the carbonyl reductase mutant are positions 82, 88, 121, 138, 142, 190, 193, 204 and 206 of the amino acid sequence as shown in SEQ ID NO: 1;
(4) the mutation sites of the carbonyl reductase mutant are positions 82, 88, 121, 138, 142, 190, 193, 206 and 207 of the amino acid sequence as shown in SEQ ID NO: 1;
(5) the mutation sites of the carbonyl reductase mutant are positions 82, 88, 121, 138, 142, 190, 193, 201, 206 and 207 of the amino acid sequence as shown in SEQ ID NO: 1;
(6) the mutation sites of the carbonyl reductase mutant are positions 82, 88, 121, 138, 142, 190, 192, 193, 204 and 206 of the amino acid sequence as shown in SEQ ID NO: 1;
(7) the mutation sites of the carbonyl reductase mutant are positions 82, 88, 121, 138, 142, 190, 193, 201 and 204 of the amino acid sequence as shown in SEQ ID NO: 1.

4. The carbonyl reductase mutant of claim 2, wherein the carbonyl reductase mutant comprises one or more of the following mutations:
(1) the amino acid residue at position 82 is mutated from W to L;
(2) the amino acid residue at position 142 is mutated from R to M, F, H, or L, such as M;
(3) the amino acid residue at position 190 is mutated from A to V;
And (4) the amino acid residue at position 193 is mutated from S to A;
preferably, the carbonyl reductase mutant further comprises one or more of the following mutations:
(1) the amino acid residue at position 88 is mutated from F to V, I or S, such as I or V;
(2) the amino acid residue of position 121 is mutated from V to A;
(3) the amino acid residue at position 138 is mutated from A to V or L, such as L;
(4) the amino acid residue at position 192 is mutated from R to M;
(5) the amino acid residue at position 201 is mutated from Y to F;
(6) the amino acid residue at position 204 is mutated from N to A or G, such as A;
(7) the amino acid residue at position 206 is mutated from K to H;
and (8) the amino acid residue at position 207 is mutated from K to N.

5. The carbonyl reductase mutant of claim 1, wherein the mutation sites and types of the carbonyl reductase mutant are shown in the following table:

| Mutant | Amino acid residue difference from SEQ ID NO: 1 |
|---|---|
| Mutant 19 | F88V, R142M, A190V, S193A |
| Mutant 21 | F88I, R142L, A190V, S193A |
| Mutant 23 | F88I, R142M, A190V, S193A |
| Mutant 25 | F88I, R142F, A190V, S193A |
| Mutant 27 | F88V, R142L, A190V, S193A |
| Mutant 29 | F88S, R142H, A190V, S193A |
| Mutant 31 | F88V, A138V, R142L, A190V, S193A |
| Mutant 33 | F88I, A138L, R142I, A190V, S193A |
| Mutant 35 | F88I, A138L, R142F, A190V, S193A |
| Mutant 37 | F88I, A138L, R142L, A190V, S193A |
| Mutant 39 | F88V, A138L, R142L, A190V, S193A |
| Mutant 41 | F88I, A138L, R142M, A190V, S193A |
| Mutant 43 | F88V, A138L, R142M, A190V, S193A |
| Mutant 45 | W82L, F88V, A138L, R142M, A190V, S193A |
| Mutant 49 | W82L, F88V, V121A, A138L, R142M, A190V, S193A |
| Mutant 51 | W82L, F88V, V121A, A138L, R142M, A190V, S193A, |
| | Y201F |
| Mutant 53 | W82L, F88V, V121A, A138L, R142M, A190V, S193A, |
| | N204A, K206H |
| Mutant 55 | W82L, F88V, V121A, A138L, R142M, A190V, S193A, |
| | N204G, K206H |
| Mutant 57 | W82L, F88V, V121A, A138L, R142M, A190V, S193A, |
| | K206H, K207N |
| Mutant 59 | W82L, F88I, V121A, A138L, R142M, A190V, S193A, |
| | K206H, K207N |
| Mutant 61 | W82L, F88I, V121A, A138L, R142M, A190V, S193A, |
| | Y201F, K206H, K207N |
| Mutant 63 | W82L, F88V, V121A, A138L, R142M, A190V, R192M, |
| | S193A, N204A, K206H, |
| Mutant 65 | W82L, F88V, V121A, A138L, R142M, A190V, S193A, |
| | Y201F, N204A |

6. A preparation method for the compound shown in Formula II, comprising the following steps of: in a liquid reaction system, the compound shown in formula I being subjected to the reduction reaction as shown in the following formula in the presence of coenzyme and the carbonyl reductase mutant of claim 1; 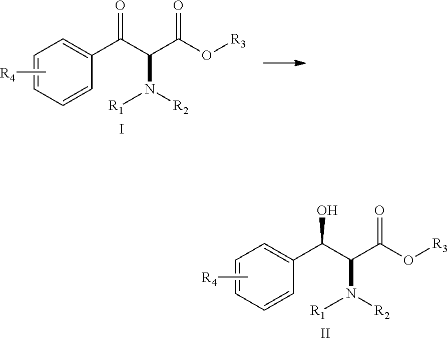
R₁ is H, 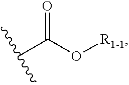
or benzyl;
R₁₋₁ is C₁-C₆ alkyl or benzyl;
R₂ is H, 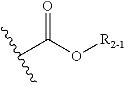
or benzyl;
R₂₋₁ is C₁-C₆ alkyl or benzyl;
R₃ is 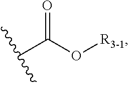
wherein R₃₋₁ is C₁-C₆ alkyl;
R₄ is H, NO₂, halogen, C₁-C₆ alkyl, C₁-C₆ alkoxy, or C₁-C₆ alkyl substituted sulfonyl.

7. The preparation method for the compound shown in Formula II of claim 6, wherein the reduction reaction meets one or more of the following conditions:
(1) the halogen is F, Cl, Br or I;
(2) the C₁-C₆ alkyl is a C₁-C₄ alkyl, such as methyl, ethyl, n-propyl, isopropyl, n-butyl, sec-butyl, isobutyl or tert-butyl;
(3) the coenzyme is a reducing coenzyme and/or an oxidative coenzyme; the preferred oxidative coenzyme is NAD⁺ and/or NADP⁺; the preferred reducing coenzyme is NADH and/or NADPH;
(4) the mass ratio of the coenzyme to the compound shown in Formula I is 1: (1-100), preferably 1: (50-100), for example, 1:100 or 1:75;
(5) the liquid reaction system comprises an enzyme for coenzyme regeneration and a co-substrate for coenzyme regeneration; the enzyme used for coenzyme regeneration is preferably one or more selected from alcohol dehydrogenase, formate dehydrogenase, and glucose dehydrogenase, such as glucose dehydrogenase; the co-substrate is preferably one or more selected from isopropanol, glucose, and ammonium formate, such as glucose; preferably, the liquid reaction system further comprises a buffer solution such as a phosphate buffer solution; the preferred phosphate buffer is 0.1M phosphate buffer;
(6) the reaction temperature for the reduction reaction is 10° C.-50° C., preferably 25° C.-35° C., for example, 30° C.;
(7) the reaction time of the reduction reaction is 0.1-72 hours, preferably 3-24 hours;
(8) the pH of the reduction reaction is 6-10, preferably 7.0-9.0, such as 7.5-8.0;
and, (9) the carbonyl reductase mutant is added to the reduction reaction in the form of free enzyme, immobilized enzyme, bacterial powder or bacterial form enzyme, preferably in bacterial form enzyme.

8. The preparation method for the compound shown in Formula II of claim 7, wherein the reduction reaction meets one or more of the following conditions:
(1) R₁ is H, 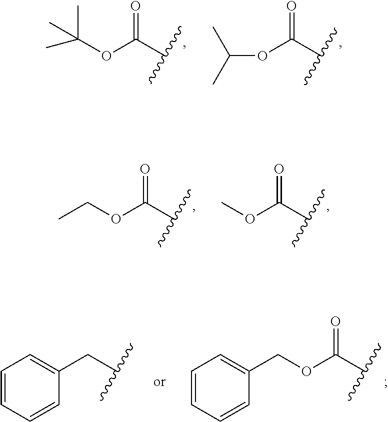
(2) R₂ is H, 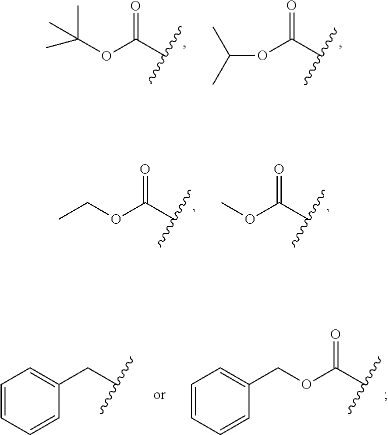
(3) R₃ is 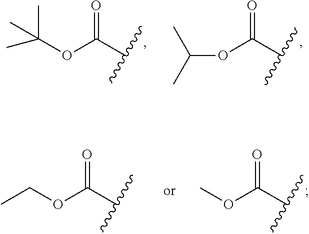
(4) R₄ is H, NO₂, F, Cl, Br, I, methyl, methoxy or 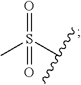 [SMILES: *S(C)(=O)=O]
(5) the liquid reaction system further comprises a cosolvent, the cosolvent is preferably selected from one or more of dimethyl sulfoxide, isopropanol, and methylbenzene, preferably dimethyl sulfoxide;
(6) the mass concentration of co-substrate in the liquid reaction system is 5-30%, preferably 5-20%, for example, 16%, 8%, or 12%;
(7) the mass concentration of enzyme used for coenzyme regeneration in the liquid reaction system is 1-10%, preferably 1%-5%, for example, 2.5%, 1.6%, or 2.3%;
(8) after the reduction reaction is completed, a post-processing step is further incorporated; preferably, the post-processing step comprises: adding an organic solvent to the liquid reaction system, heating, filtering the bacterial body, extracting, washing the organic phase with water, drying, and filtering to concentrate the organic layer to obtain a compound shown in Formula II;
preferably, R₁ is H, R₂ is 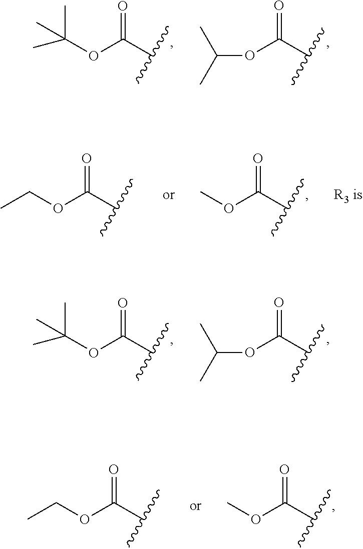
and R₄ is F, Cl or Br.

9. The preparation method for the compound shown in Formula II of claim 8, wherein the reduction reaction meets one or more of the following conditions:
(1) the compound as shown in Formula I is 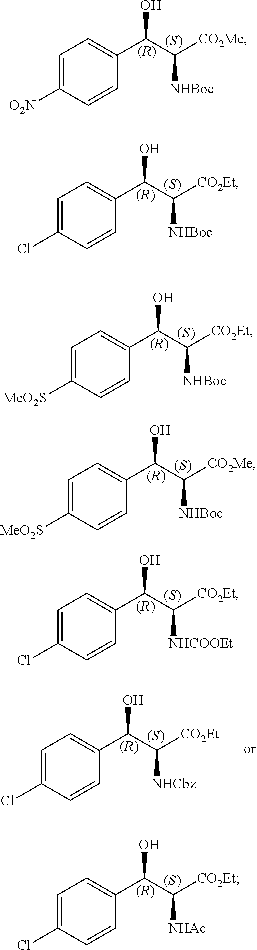
(2) the compound as shown in Formula II is 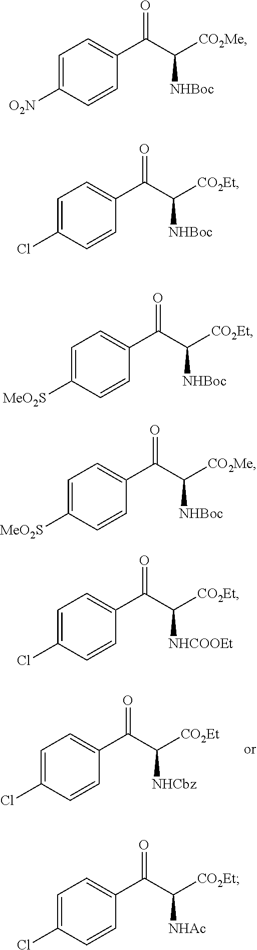
(3) the mass concentration of the cosolvent in the liquid reaction system is 10%-50%, preferably 20%-30%, for example, 30%, 29%, or 28%;
(4) the organic solvent is an ester solvent, an ether solvent, an alcohol solvent, an aromatic solvent, or a chlorinated alkane solvent; the preferred ester solvent is ethyl acetate or isopropyl acetate; the preferred ether solvent is methyl tert-butyl ether or 2-methyltetrahydrofuran; the preferred alcohol solvent is n-butanol; the preferred aromatic solvent is methylbenzene; the preferred chlorinated alkane solvent is dichloromethane;
(5) the heating temperature is 60° C.;
(6) the heating time is 1 hour;
(7) the water used for washing comprises pure water and water containing inorganic salts, for example, pure water and/or 5% saline solution;
and (8) the drying method is using desiccant for drying; the preferred desiccant is anhydrous sodium sulfate;
preferably, the post-processing step comprises: adding methyl tert-butyl ether or ethyl acetate to the reaction solution of the reduction reaction, heating, filtering the bacterial body, and extracting, washing the organic phase with pure water and 5% saline solution, drying with anhydrous sodium sulfate, and filtering to concentrate the organic layer to obtain the compound shown in Formula II.

10. An application of the carbonyl reductase mutant of claim 1 in reducing carbonyl;


SEQUENCE LISTING (2 sequences)

SEQ ID NO: 1 - protein, 249 aa, Exiguobacterium acetylicum
MKYTVITGASSGIGYETAKLLAGKGKSLVLVARRTSELEKLRDEVKQISPDSDVILKSVDLADNQNVHDLYEGLKELDIETWINNAGFGDFDLVQDIELGKIEKMLRLNIEALTILSSLFVRDHHDIEGTTLVNISSAGGYRIVPNAVTYCATKFYVSAYTEGLAQELQKGGAKLRAKVLAPAATETEFADRSRGEAGFDYSKNVKKYHTAAEMAGFLHQLIESDAIVGIVDGETYEFELRGPLFNYAG

SEQ ID NO: 2 - DNA, 750 bp, Exiguobacterium acetylicum
  source 1..750: mol_type = genomic DNA
atgaaatacaccgttatcaccggtgcttcttctggtatcggttacgaaaccgctaaactgctggctggtaaaggtaaatctctggttctggttgctcgtcgtacctctgaactggaaaaactgcgtgacgaagttaaacagatctctccggactctgacgttatcctgaaatctgttgacctggctgacaaccagaacgttcacgacctgtacgaaggtctgaaagaactggacatcgaaacctggatcaacaacgctggtttcggtgacttcgacctggttcaggacatcgaactgggtaaaatcgaaaaaatgctgcgtctgaacatcgaagctctgaccatcctgtcttctctgttcgttcgtgaccaccacgacatcgaaggtaccaccctggttaacatctcttctgcgggtggttaccgtatcgttccgaacgctgttacctactgcgctaccaaattctacgtttctgcttacaccgaaggtctggctcaggaactgcagaaaggtggtgctaaactgcgtgctaaagttctggctccggctgctaccgaaaccgaattcgctgaccgtagccgtggtgaagctggtttcgactactctaaaaacgttaaaaaataccacaccgctgctgaaatggctggtttcctgcaccagctgatcgaatctgacgctatcgttggtatcgttgacggtgaaacctacgaattcgaactgcgtggtccgctgttcaactacgctggttaa